(19) United States
(12) Patent Application Publication
(10) Pub. No.: US 2013/0023722 A1
(43) Pub. Date: Jan. 24, 2013

(54) TACTILE USER DEVICES AND METHODS

(71) Applicant: Marie Kruk, Lloydminster (CA)

(72) Inventor: Marie Kruk, Lloydminster (CA)

(21) Appl. No.: 13/549,587

(22) Filed: Jul. 16, 2012

Related U.S. Application Data
(60) Provisional application No. 61/508,905, filed on Jul. 18, 2011.

(51) Int. Cl. A61M 21/02; A63H 3/36; A63H 3/04; A61H 39/08; A61M 5/168

(52) U.S. Cl. 600/27; 606/189; 604/67; 446/72

(57) ABSTRACT

A patient seeking complementary and alternative medicine must visit a practitioner which is not always convenient or possible. It would be beneficial to provide a patient with a simple means of deriving benefit from such physical CAM techniques in a non-intimidating manner whereby the means has familiarity associated with it. Even with conventional medicine children, infants, toddlers, and babies require medical treatment and experience fear and/or anxiety arising from visiting the medical practitioner or with the treatment. According to embodiments of the invention provide patients are provided means to exploit CAM techniques away from the practitioner or allow patients to not associate activities with a medical treatment but another activity, such as playing in the instance of a child. Variations of the means allow medical monitoring activities to be similarly associated with familiar objects and non-traumatic activities.

### CROSS-REFERENCE TO RELATED APPLICATIONS

This patent application claims the benefit of U.S. Provisional Patent Application 61/508,905 filed Jul. 18, 2011 entitled “Birthing Bobble.”

### FIELD OF THE INVENTION

The present invention relates to pain relief and in particular to devices and methods to engage individuals with familiar objects to support providing pain relief.

### BACKGROUND OF THE INVENTION

Industry analysts estimate that more than 1.5 billion people around the world suffer from chronic pain; making it the number one reason patients seek medical care. In fact, studies have shown that pain leads to more than 50 million lost workdays each year. The cost of pain, including medical bills and lost workdays, is estimated at $100 billion per year. Within the United States alone the market for pain-management therapeutics was estimated to generate more than $40 billion in 2011 and is predicted to grow to $60 billion by 2015. This market includes treatments for a wide range of conditions including postoperative pain, cancer pain, arthritis pain, migraine pain, neuropathic pain, and back pain.

Pain management (also called pain medicine or algiatry) is a branch of medicine employing an interdisciplinary approach for easing the suffering and improving the quality of life of those living with pain. The typical pain management team includes medical practitioners, clinical psychologists, physiotherapists, occupational therapists, and nurse practitioners. Pain sometimes resolves promptly once the underlying trauma or pathology has healed, and is treated by one practitioner, with drugs such as analgesics and (occasionally) anxiolytics. Effective management of long term pain, however, frequently requires the coordinated efforts of the management team.

Medicine treats injury and pathology to support and speed healing; and treats distressing symptoms such as pain to relieve suffering during treatment and healing. When a painful injury or pathology is resistant to treatment and persists, when pain persists after the injury or pathology has healed, and when medical science cannot identify the cause of pain, the task of medicine is to relieve suffering. Treatment approaches to long term pain include pharmacologic measures, such as opioids, nonsteroidal anti-inflammatory drugs, acetaminophen, local anesthetics, tricyclic antidepressants, anticonvulsants, patient-controlled dosing, interventional procedures, physical therapy, physical exercise, application of ice and/or heat, implants, electrical stimulation, and psychological measures, such as biofeedback and cognitive behavioral therapy. Accordingly today there is high demand for more efficient drugs, delivery systems, and medical devices for pain treatment is driving the pain-management market. The need to address acute and chronic pain will rise alongside an increasingly aging population. Other factors affecting the future of the pain-management segment include a growing number of surgical procedures being performed, changing lifestyles, and increasing incidences of cancer.

Pain medications are in many instances simple in that the patient may self-medicate according to the pain or follow a predetermined regime without the need for continuous medical team involvement; rather it becomes intermittent such as at the end of a drug regime or when the patient requires a repeat prescription. However, many pain medications, while effective, cause unwanted side effects. Opioids, the largest pain-therapy class, are widely known to cause a number of side effects such as nausea, vomiting, constipation, and somnolence. They can also cause severe respiratory depression. Nonsteroidal anti-inflammatory drugs (NSAIDs) have been shown to cause gastrointestinal upset, kidney and liver damage, and they can increase cardiovascular risks. Additionally a significant number of individuals either do not want to exploit drug treatments, cannot afford the drug treatments, have cultural or religious barriers to some or all drug treatments, or cannot take them due to a compounding condition such as pregnancy or cancer for example in order to protect the unborn infant or prevent drug reactions respectively.

Accordingly, there are wide varieties of what are known as “alternative” medicines”, which is a blanket term for any healing practice “that does not fall within the realm of conventional medicine.” These are in many instances also referred to as complementary as they do not impact “conventional” medicines or treatments. Typically these are based upon on historical and/or cultural traditions and in some ethnic groups, countries, etc are the dominant means of pain management. Additionally in many instances patients may combine them, such as for example approximately one third of cancer patients in the western world will exploit some form of complementary and alternative medicine. Alternative medicine may vary from country to country and in some jurisdictions where alternative medical practices are sufficiently widespread they may license and regulate them. In other jurisdictions complementary and alternative medicine is mainly in the hands of physicians, for example Germany and Austria, whilst some estimates suggest that at least half of American alternative practitioners are physicians.

Alternative medicine is frequently grouped with complementary medicine or integrative medicine, which, in general, refers to the same interventions when used in conjunction with mainstream techniques, under the umbrella term complementary and alternative medicine, or CAM. Some researchers in alternative medicine object to this categorization, preferring to emphasize differences of approach, but nevertheless use the term CAM, which has become standard. Critics maintain that the terms “complementary” and “alternative medicine” are deceptive euphemisms meant to give an impression of medical authority.

CAM methods are diverse in their foundations and methodologies and may incorporate or base themselves on traditional medicine, folk knowledge, spiritual beliefs, or newly conceived approaches to healing. Although heterogeneous, the major CAM systems have many common characteristics, including a focus on individualizing treatments, treating the whole person, promoting self-care and self-healing, and recognizing the spiritual nature of each individual. In addition, many CAM systems have characteristics commonly found in mainstream healthcare, such as a focus on good nutrition and preventive practices. Unlike mainstream medicine, CAM often lacks or has only limited experimental and clinical study; however, scientific investigation of CAM is beginning to address this knowledge gap. Thus, boundaries between CAM and mainstream medicine, as well as among different CAM systems, are often blurred and are constantly changing. The U.S. National Center for Complementary and Alternative Medicine (NCCAM)

1. Whole medical systems: cut across more than one of the other groups; examples include Traditional Chinese medicine, Naturopathy, Homeopathy, and Ayurveda

2. Mind-body medicine: takes a holistic approach to health that explores the interconnection between the mind, body, and spirit. It works under the premise that the mind can affect “bodily functions and symptoms”

3. Biology-based practices: use substances found in nature such as herbs, foods, vitamins, and other natural substances

4. Manipulative and body-based practices: feature manipulation or movement of body parts, such as is done in chiropractic and osteopathic manipulation

5. Energy medicine: is a domain that deals with putative and verifiable energy fields such as biofield therapies which are intended to influence energy fields that are purported to surround and penetrate the body and bioelectromagnetic-based therapies use verifiable electromagnetic fields, such as pulsed fields, alternating-current, or direct-current fields in an unconventional manner.

One common CAM is reflexology, or zone therapy, is an alternative medicine involving the physical act of applying pressure to the feet, hands, or ears with specific thumb, finger, and hand techniques without the use of oil or lotion. It is based on what reflexologists define as a system of zones and reflex areas that they say reflect an image of the body on the feet and hands, with the premise that such work effects a physical change to the body.

Another common CAM is acupuncture which originated in Ancient China and treats patients by insertion and manipulation of solid, generally thin needles in the body. According to traditional Chinese medicine, bodily functions are regulated by the flow of an energy-like entity called qi. Acupuncture aims to correct imbalances in the flow of qi by stimulation of anatomical locations on or under the skin called acupuncture points, most of which are connected by channels known as meridians.

At present a patient seeking to use such physical CAM techniques must visit a practitioner which is not always convenient or possible. In many instances it would be beneficial to provide a patient with a simple device allowing them to derive benefit from such physical CAM techniques in a non-intimidating manner whereby the device has familiarity associated with it. Even in conventional medicine children, infants, toddlers, and babies require medical treatment and in many instances a fear associated with the medical treatment and/or anxiety arising from visiting the medical practitioner etc result in reduced or no treatment as the patient refuses to take the medicine or is physically sick immediately thereafter.

In many instances therefore it would be beneficial to provide patients and individuals with devices that either allow them to exploit these CAM techniques away from the practitioner or allow the patient to not associate an activity with a medical treatment but another activity, such as playing in the instance of a child. Beneficially embodiments of the invention provide devices for such instances.

Other aspects and features of the present invention will become apparent to those ordinarily skilled in the art upon review of the following description of specific embodiments of the invention in conjunction with the accompanying figures.

### SUMMARY OF THE INVENTION

It is an object of the present invention to provide devices for pain relief and in particular to devices and methods to engage individuals with familiar objects to support providing pain relief.

In accordance with an embodiment of the invention there is provided a device comprising:
an outer shell formed from a first material characterized by a first resilience and having a first predetermined portion providing an association for an individual to a familiar object and a second predetermined portion providing a predetermined orientation of the device to a first predetermined portion of the human body when placed against a second predetermined portion of the human body; and
at least one member of a plurality of members, the at least one member formed from a second material characterized by a second resilience greater than the first resilience and being positioned in a first predetermined position within the device so as to engage a predetermined portion of the first predetermined portion of the human body.

In accordance with an embodiment of the invention there is provided a device comprising:
an outer shell formed from a first material characterized by a first resilience and having a first predetermined portion providing an association for an individual to a familiar object and a second predetermined portion providing a predetermined orientation of the device to a first predetermined portion of the human body when placed against a second predetermined portion of the human body;
at least one member of a plurality of members, the at least one member formed from a second material characterized by a second resilience greater than the first resilience and being positioned in a first predetermined position within the device so as to engage a predetermined portion of the first predetermined portion of the human body; and
at least one needle disposed in a predetermined position with respect to the at least one member of the plurality of members.

In accordance with an embodiment of the invention there is provided a device comprising:
an outer shell formed from a first material characterized by a first resilience and having a first predetermined portion providing an association for an individual to a familiar object and a second predetermined portion providing a predetermined orientation of the device to a first predetermined portion of the human body when placed against a second predetermined portion of the human body;
at least one member of a plurality of members, the at least one member formed from a second material characterized by a second resilience greater than the first resilience and being positioned in a first predetermined position within the device so as to engage a predetermined portion of the first predetermined portion of the human body; and
at least one needle of a plurality of needles disposed in a predetermined position with respect to the at least one member of the plurality of members;
a pressure activated sensor;
a microcontroller connected to the pressure activated sensor; and
a microfluidic delivery system disposed within the outer body connected to the needle and to a reservoir containing a fluid, the microfluidic delivery system controlled by the microcontroller.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 depicts pain relief methods according to the prior art;

FIG. 2 depicts pressure points on the hands and their associations with the rest of the human body;

FIGS. 3A and 3B depicts acupuncture points on the human hand,

FIG. 4 depicts a pain relief bobble according to an embodiment of the invention;

FIG. 5 depicts a pain relief bobble according to an embodiment of the invention;

FIG. 6 depicts a pain relief bobble according to an embodiment of the invention;

FIG. 7 depicts a pain relief bobble according to an embodiment of the invention;

FIGS. 8A and 8B depict pain relief bobbles incorporating needles according to embodiments of the invention;

FIG. 9 depicts a pain relief bobble incorporating a spray medication injection device disguised within the bobble;

FIG. 10 depicts a pain relief bobble incorporating a medication injection device disguised within the bobble;

FIG. 11 depicts a bobble incorporating a blood glucose testing device disguised within the bobble;

FIG. 12 depicts a bobble incorporating an optical blood glucose testing device disguised within the bobble;

FIG. 13 depicts a simplified schematic of the electronics for an optical blood glucose testing device according to FIG. 12;

FIG. 14 depicts a pain relief bobble according to an embodiment of the invention;

FIG. 15 depicts a pain relief bobble according to an embodiment of the invention;

FIG. 16 depicts a pain relief bobble according to an embodiment of the invention;

FIG. 17 depicts a hand massage wristband fixture for use in association with a pain relief bobble according to an embodiment of the invention;

FIG. 18 depicts a hand massage wristband fixture for use in association with a pain relief bobble according to an embodiment of the invention; and

FIG. 19 depicts a pain relief bobble according to an embodiment of the invention wherein elements of the pain relief bobble may be customized to the user.

### DETAILED DESCRIPTION

The present invention is directed to pain relief and in particular to devices and methods to engage individuals with familiar objects to support providing pain relief.

The ensuing description provides exemplary embodiment(s) only, and is not intended to limit the scope, applicability or configuration of the disclosure. Rather, the ensuing description of the exemplary embodiment(s) will provide those skilled in the art with an enabling description for implementing an exemplary embodiment. It being understood that various changes may be made in the function and arrangement of elements without departing from the spirit and scope as set forth in the appended claims.

A “user” or “patient” as used herein and through this disclosure refers to, but is not limited to, a person who utilizes a device according to an embodiment of the invention to derive benefits from their utilization.

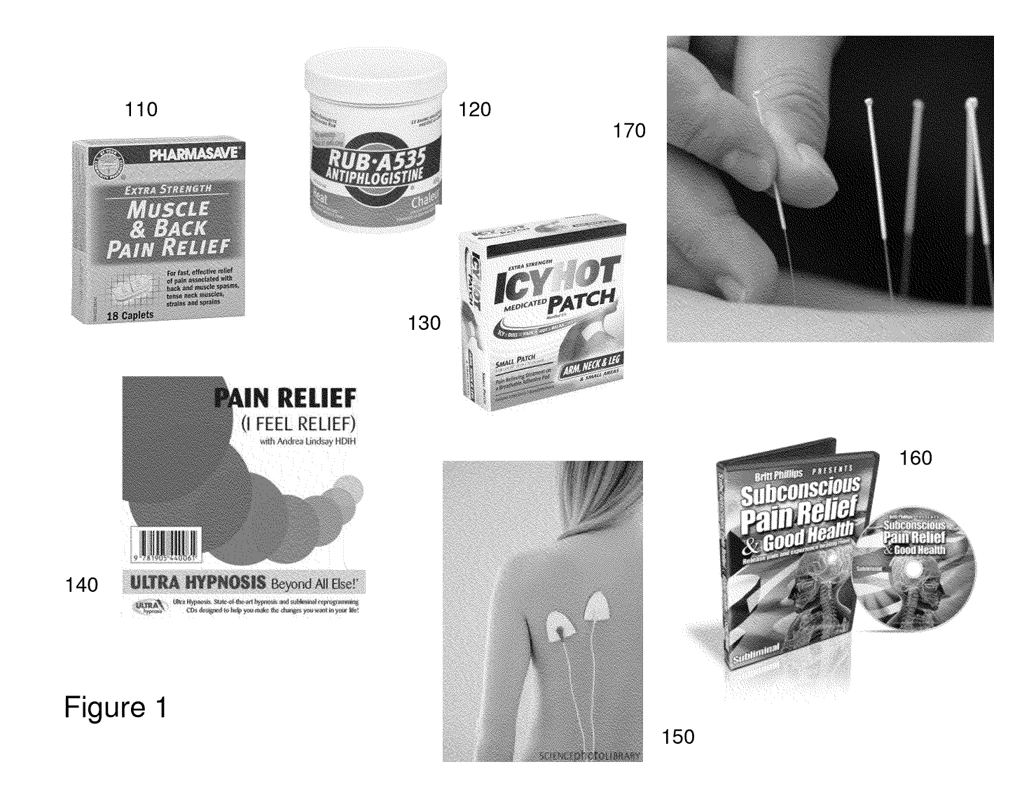

Referring to FIG. 1 there are depicted pain relief methods according to the prior art including over-the-counter medicine 110, heat releasing compound 120, a patch 130 that starts by cooling then heating, hypnosis 140, electrical therapy 150, subconscious therapy 160, and acupuncture 170. Over-the-counter medicine 110 and prescription medicines, not shown for clarity but generally stronger, target physiological processes such as blocking neural pain transmitters and form the primary thrust of conventional medicine. Heat releasing compound 120 and patch 130 are common treatments for patients suffering aches or pain within their limbs, neck and back. The other techniques form part of the CAM regimen adopted by many patients.

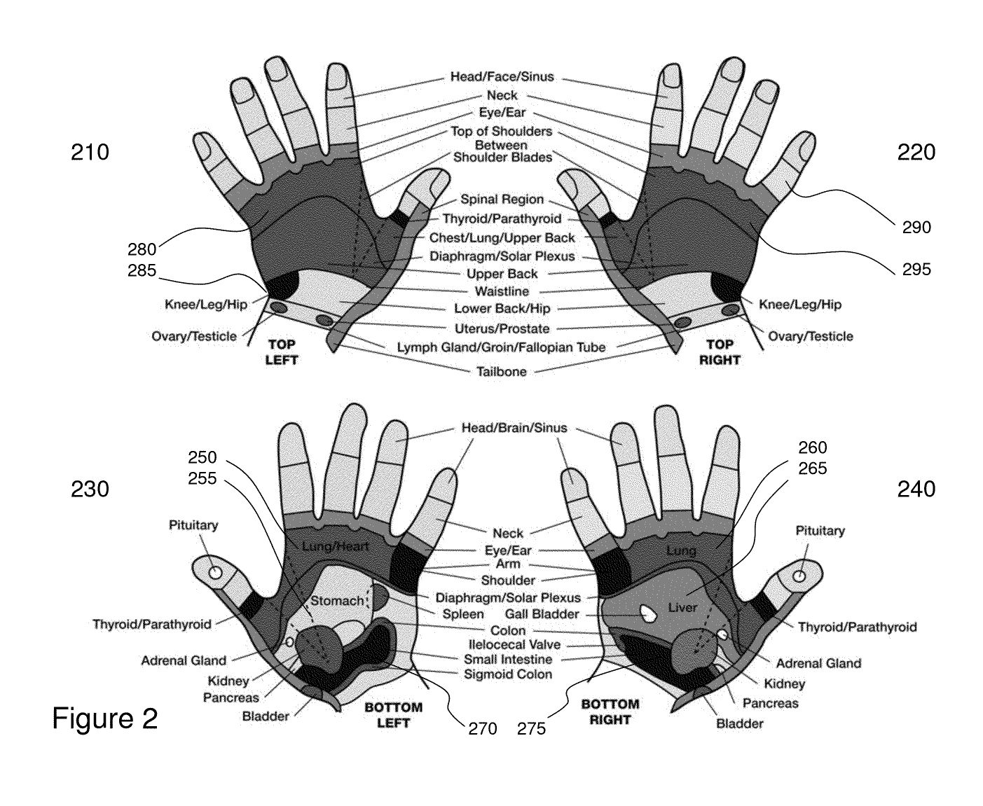

Referring to FIG. 2 there are depicted first to fourth images 210 to 240 respectively depicting the left back, right back, left front (palm), and right front (palm) views of patient's hands respectively and indicating where reflexology practitioners recognise pressure points on the hands and their associations with the rest of the human body. Also highlighted are first to eighth regions 260 through 295 respectively as outlined below in Table 1.

TABLE 1
Some Reflexology Associations of Regions
of Human Hands to Human Body

| Region | Area of Patient Hand | Area of Human Body |
|---|---|---|
| 260 | Right - bottom - base of fingers | Lung |
| 265 | Right - bottom - palm | Liver |
| 270 | Left - bottom - below thumb | Small intestine |
| 275 | Right - bottom - below thumb | Small intestine |
| 280 | Left - top - across knuckles | Top of shoulders |
| 285 | Left - top - wrist/little finger area | Knee/Leg/Hip |
| 290 | Right - top- middle of finger | Eye/Ear |
| 295 | Right - top - across knuckles | Top of shoulders |

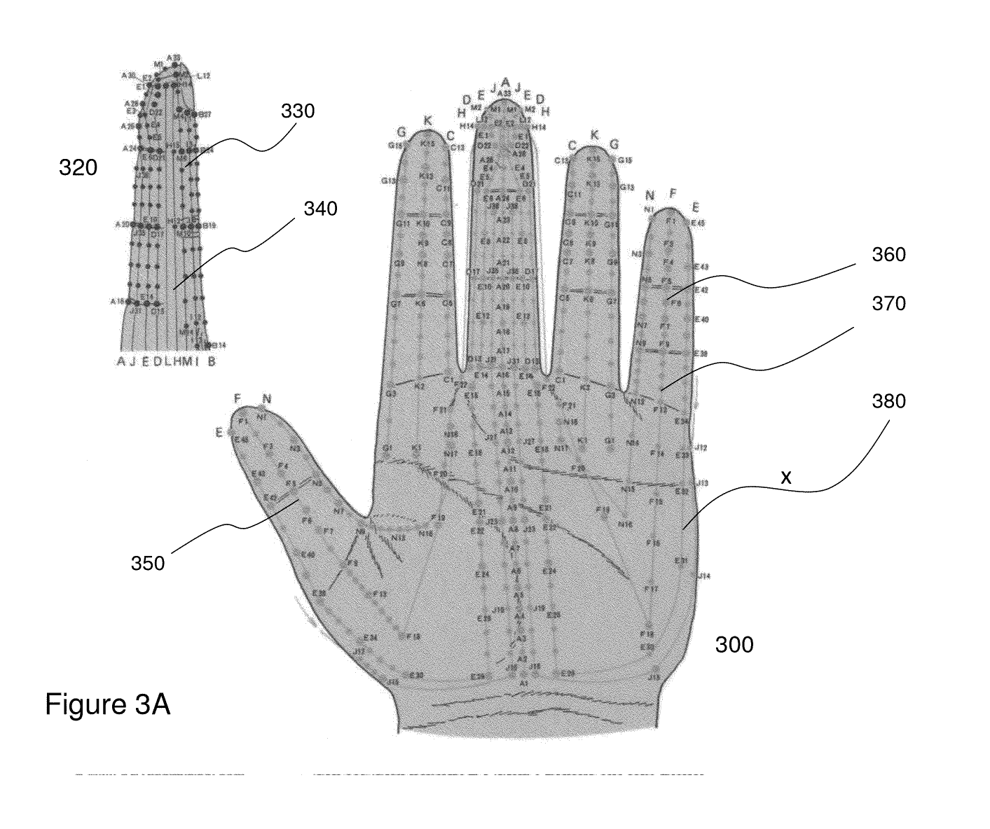

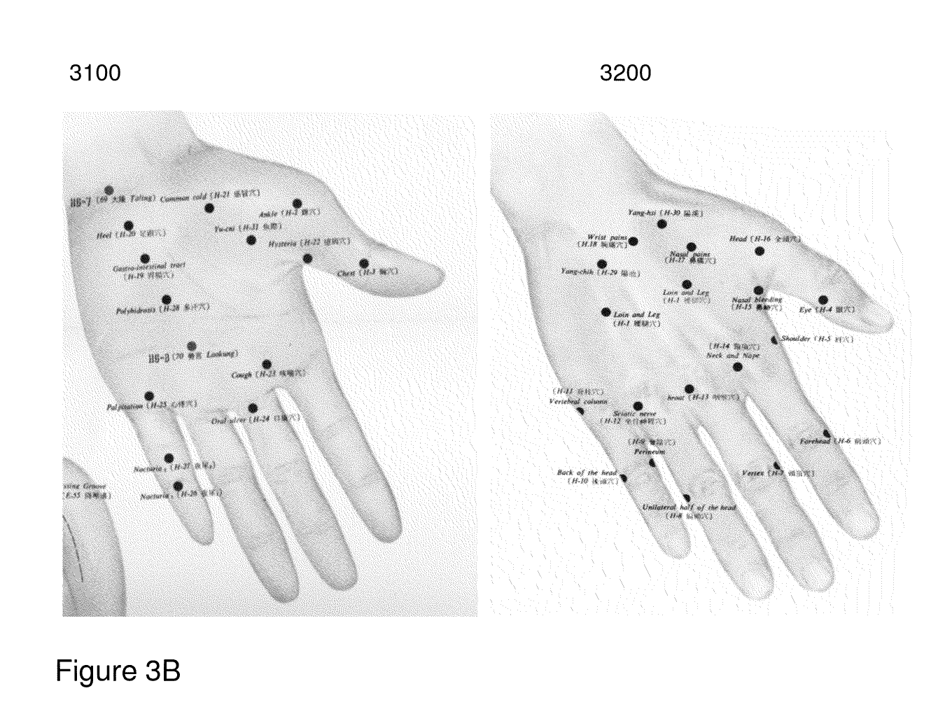

FIG. 3 depicts an Ancient Chinese acupuncture points on the human hand in first image 300 and finger 320 depicting meridians 340, 350, 380 for example and acupuncture points 330, 360 and 370. Despite the large number of points depicted in the Ancient Chinese texts and these images today approximately 360 points are generally recognized all over the human body. Accordingly in FIG. 3B palm and back images 3100 and 3200 respectively are depicted identifying the dominant acupuncture points which are shown as 15 and 19 respectively for the palm and back of the hand.

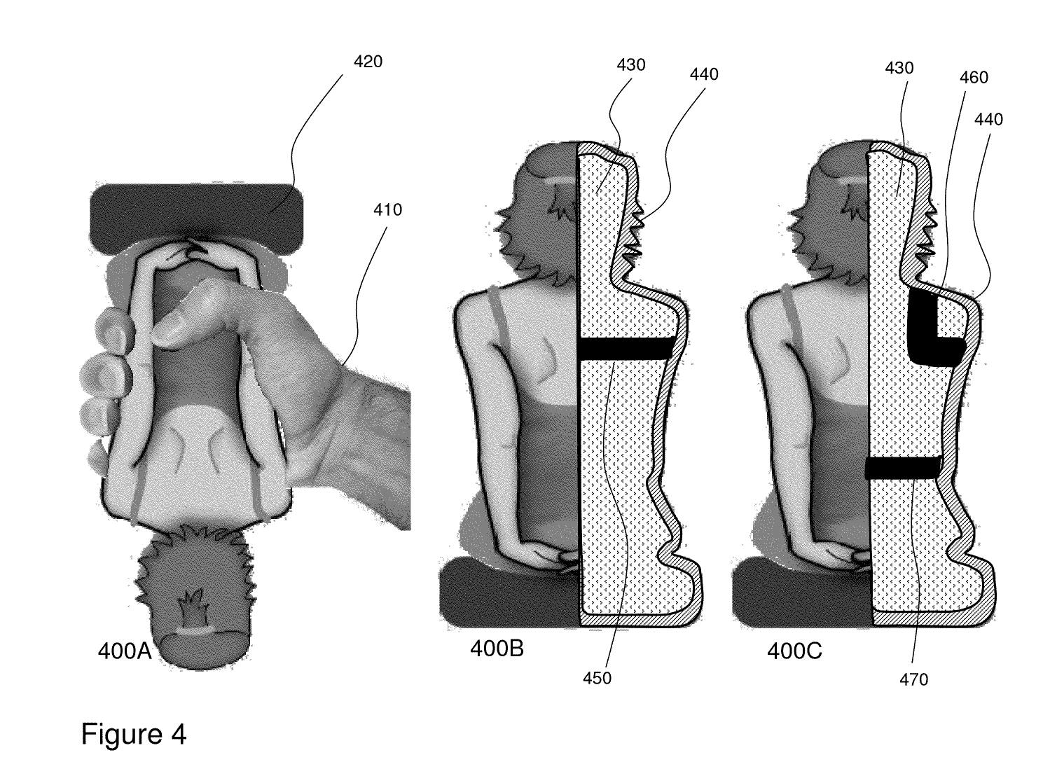

Now referring to FIG. 4 there are depicted first to third images 400A to 400C respectively of a pain relief bobble 420 according to an embodiment of the invention. Within first image 400A the pain relief bobble 420 is shown inserted into a patient's hand 410. As evident the outer shape of the pain relief bobble 420 has been designed to orientate and locate within the patients hand 410. In this instance the pain relief bobble 420 is depicted upside down but it would be evident to one skilled in the art that it may equally be designed for a more traditional orientation with the head upwards. In second image 400B there is depicted a cross-sectional view of a first embodiment of the pain relief bobble 420 according to an embodiment of the invention wherein an outer shell 440 is depicted which is filled with a filler 430. Disposed within the filler 430 is a ring 450 extending through the width of the body. Accordingly where filler 430 and outer body 440 are implemented with materials having a combined resiliency allowing the pain relief bobble 420 to compress under an increasing grip of the patient the ring 450 is implemented with an increased resiliency such that pressure is then applied to the patients hand at the location on their hand that they are gripping the section with ring 450.

Likewise third image 400B with a cross-section of a different implementation of the pain relief bobble 420 according to an embodiment of the invention there are disposed first and second resilient members 460 and 470 which provide pressure to two different points on the patient's hand. First resilient member 460 provides a different resilience than second resilient member 470 even where they are made from the same material as first resilient member is anchored only to the outer shell 440 whereas second resilient member 470 extends through the body to the other side and hence pressure applied from the opposite side of the pain relief bobble 420 is also transmitted to the patients palm according to the properties of material forming the second resilient member 470.

It would be evident to one skilled in the art that the pain relief bobble 420 may be provided in a variety of manners to appear familiar or reassuring to the patient. For example, a mother giving birth to a boy may have a male baby shaped pain relief bobble 420, a mother giving birth to a girl a girl shaped pain relief bobble 420, a girl may have a girl shaped pain relief bobble 420, a boy may have a racing car shaped pain relief bobble 420. Accordingly it would be evident that the overall visual appearance of the pain relief bobble 420 may be varied to include people, historical figures, objects, fictional characters, animals, etc. Irrespective of the shape the pain relief bobble 420 would be molded to provide a grip to the patient that orientated the resilient members to the correct location in the patient's hand. Accordingly a pain relief bobble 420 for a child may be dimensioned narrower than that of an adult, and an adult man's version differently to an adult woman's version.

It would be evident to one skilled in the art that the outer shell 440 may be provided from a rubber or plastic material for example providing compliance together with the ability to be molded/cast for example to the desired shape plus support pigmentation etc as applied to increase the objects familiarity to the patient. Filler 430 may be air, a liquid, foam, plastic or other material combining with the outer shell 440 to provide the required overall physical feel and resiliency to the pain relief bobble 420. Filler 430 may also be the same material as outer shell 440. Resilient member may for example be another plastic, rubber or harder material providing the required resilience to the patients hand such that it does not deform to the same extent as the outer shell 440 and filler 430 thereby applying pressure to the patients hand in the region of the resilient member 450.

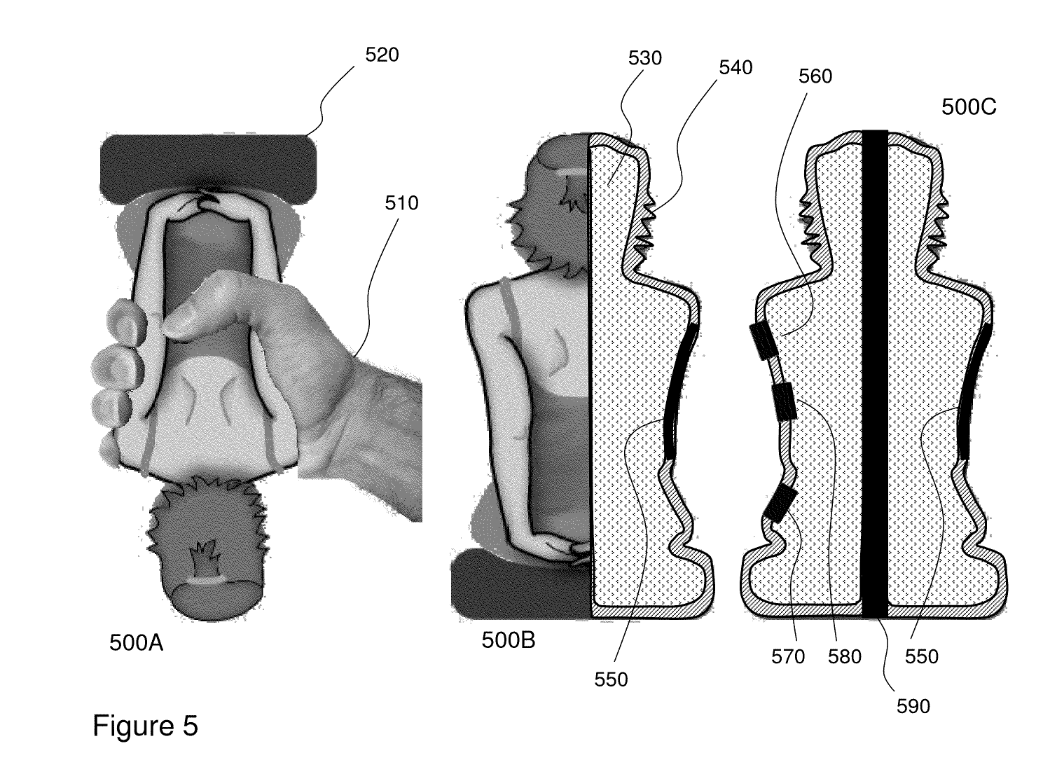

Now referring to FIG. 5 there are depicted first to third images 500A to 500C respectively of a pain relief bobble 520 according to an embodiment of the invention. Within first image 500A the pain relief bobble 520 is shown inserted into a patient's hand 510. As evident the outer shape of the pain relief bobble 520 has been designed to orientate and locate within the patients hand 510. In this instance the pain relief bobble 520 is depicted upside down but it would be evident to one skilled in the art that it may equally be designed for a more traditional orientation with the head upwards. In second image 500B there is depicted a cross-sectional view of a first embodiment of the pain relief bobble 520 wherein an outer shell 540 is depicted which is filled with a filler 530. Disposed within the outer body 540 is a region 550. Accordingly where filler 530 and outer body 540 are implemented with materials having a combined resiliency allowing the pain relief bobble 520 to compress under an increasing grip of the patient the region 550 is implemented with an increased resiliency such that pressure is then applied to the patients hand at the location on their hand that they are gripping the section with region 550.

Likewise third image 500B with a cross-section of a different implementation of the pain relief bobble 520 according to an embodiment of the invention wherein there are disposed first to third resilient members 560 to 580 respectively which provide pressure to three different points on the patient's hand opposite the region 550. First to third resilient members 560 to 580 respectively provides a different resilience than region 550 even where they are made from the same material due to their dimensional differences and by virtue of being short sections within the outer body 540 rather than a long section as in region 550. Optionally first to third resilient members 560 to 580 respectively may be different materials to region 550 but they may also be made from two or more materials themselves such that for example first resilient member 560 is formed from a first material whilst second and third resilient members 570 and 580 respectively are formed from another material. As with pain relief bobble 420 in FIG. 4 pain relief bobble 520 may be implemented in a variety of materials, finishes, colours, designs etc.

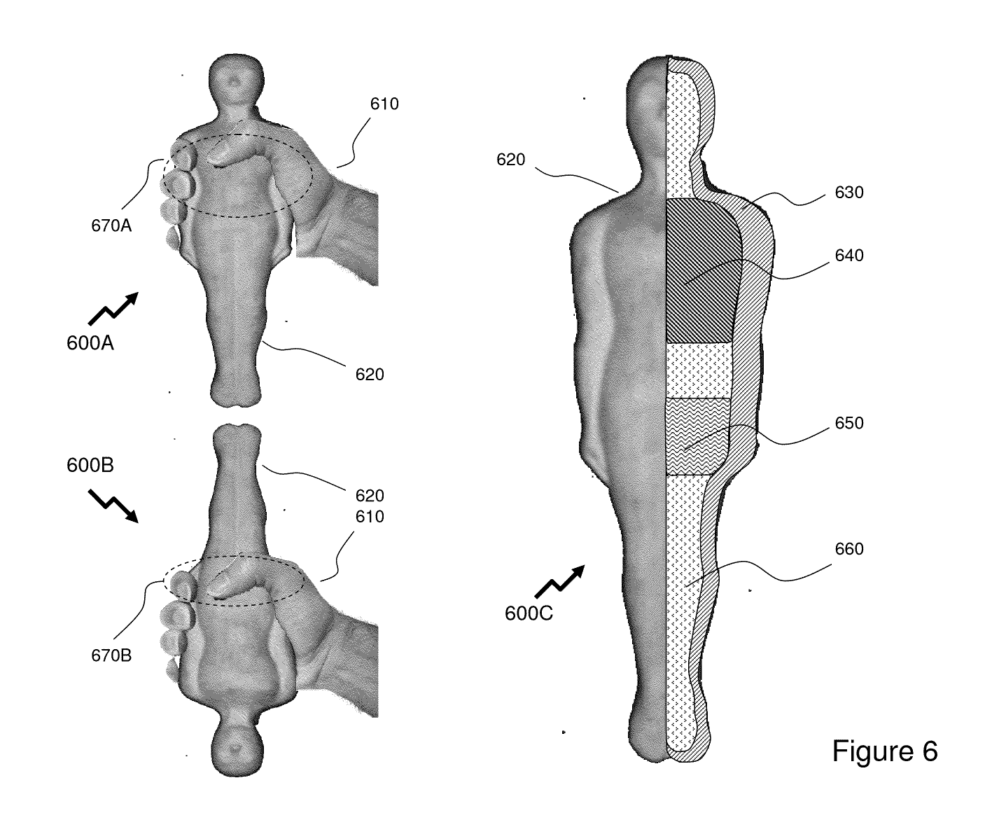

Now referring to FIG. 6 there are depicted first to third images 600A to 600C respectively for a pain relief bobble 620 according to an embodiment of the invention. As depicted in first and second images 600A and 600B respectively the pain relief bobble 620 may be gripped by the patients hand in either an upright or inverted manner. Now referring to third image 600C there is depicted a cross-section of a pain relief bobble 620 according to an embodiment of the invention comprising an outer shell 630 within which are disposed first and second resilient elements 640 and 650 within the remainder of the inner region of the pain relief bobble 620 which is filled with filler 660. As depicted first resilient member 640 provides resilience against the patients hand in first region 670A depicted in first image 600A and when the pain relief bobble is inverted the second resilient member 650 provides a different resilience against the patients hand in a smaller region 670B as depicted in second image 600B.

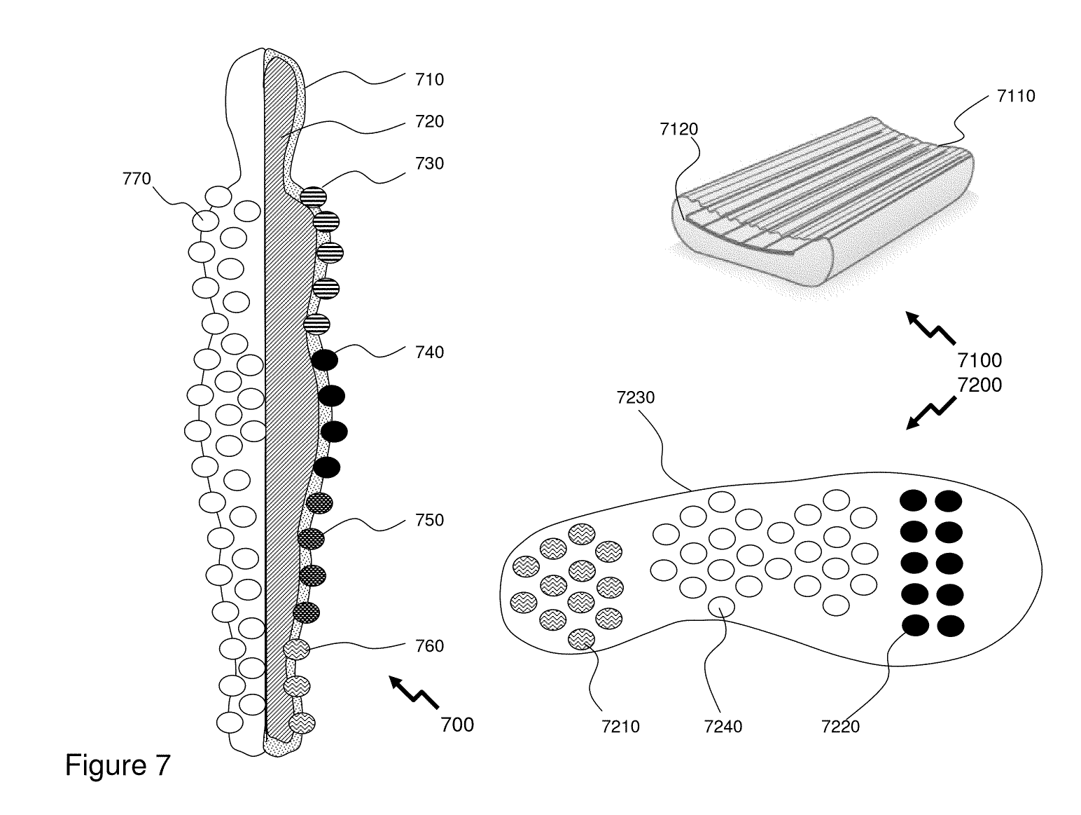

Now referring to FIG. 7 there is depicted a pain relief bobble 700 according to an embodiment of the invention in partial cross-sectional view. As shown on the left side of pain relief bobble 700 the surface has a plurality of bumps 770 in a predetermined pattern. Now referring to the partial cross-section of the pain relief bobble 700 comprises an outer shell 710 with a filler 720 and first to fourth resilient groups 730 to 770 respectively which are disposed according to a predetermined pattern. In this instance the first to fourth resilient groups 730 to 770 respectively are disposed vertically in groups along the length of the pain relief bobble. However, it would be evident that other patterns are possible including the mixing of resilient groups within the same region of the pain relief bobble 700. Each of the first to fourth resilient groups 730 to 770 respectively provides a different effective pressure to the patients hand when they squeeze the pain relief bobble 700.

It would be evident to one skilled in the art that the shape, texture, and material of each of the first to fourth resilient groups 730 to 770 respectively may be varied. For example, hard spike like elements may be employed in some regions with soft spherical surface elements of these may be intermingled with varying densities to provide different region properties. It would also be evident that the number of different resilient groups may be varied such as for example 1, 2, or 3 and that the number of each may also vary in addition to their location on the pain relief bobble. Further some may project into the filler 720 or even be provided throughout the body such that they are continuous from one side of the pain relief bobble 700 to another for example.

It would be evident to one skilled in the art that pain relief bobbles as depicted and described above in respect of FIGS. 4 through 7 are primarily aimed at reflexology with the patient gripping the pain relief bobble within their hand. However, it would be evident that other designs may be implemented without departing from the scope of the invention such that for example the user stands onto the pain relief bobble to engage pressure into predetermined regions of the sole of their foot or feet or leans against the pain relief bobble for instance such that it is against their shoulders, back, calf, arm, etc. It would evident to one skilled in the art that in such instances the objects etc used t provide a degree of familiarity with the pain relief bobble may be different to those described above. Potentially these may simply be common objects such as depicted in FIG. 7 by pillow 7100 and shoe 7200. In pillow 7100 surface elements 7110 may be employed in conjunction with resilient elements 7120 embedded within the body of the pain relief bobble. With shoe 7200 first and second resilient elements 7210 and 7220 may be provided upon a sole 7230 to provide desired pressure onto the regions of the foot.

Optionally the sole 7230 contains recesses 7240 which are designed to accept discrete resilient elements such that the patient may place the resilient elements according to the pain they are suffering rather than requiring them to purchase multiple pain relief bobbles. Such a similar approach may be employed within a pain relief bobble 700 such that a plurality of recesses are provided within which resilient elements are disposed. Potentially with a regular pattern of recesses a web of resilient elements may be applied such that the patient does not need to insert a significant number of discrete elements. Optionally a medical practitioner may elect to pre-assemble a pain relief bobble and adjust the properties and/or shape of the resilient elements according to the patient wherein factors including, but not limited to, age, sex, fragility, strength, pain type, and degree of perceived pain may be considered in establishing the desired pain relief bobble for that patient.

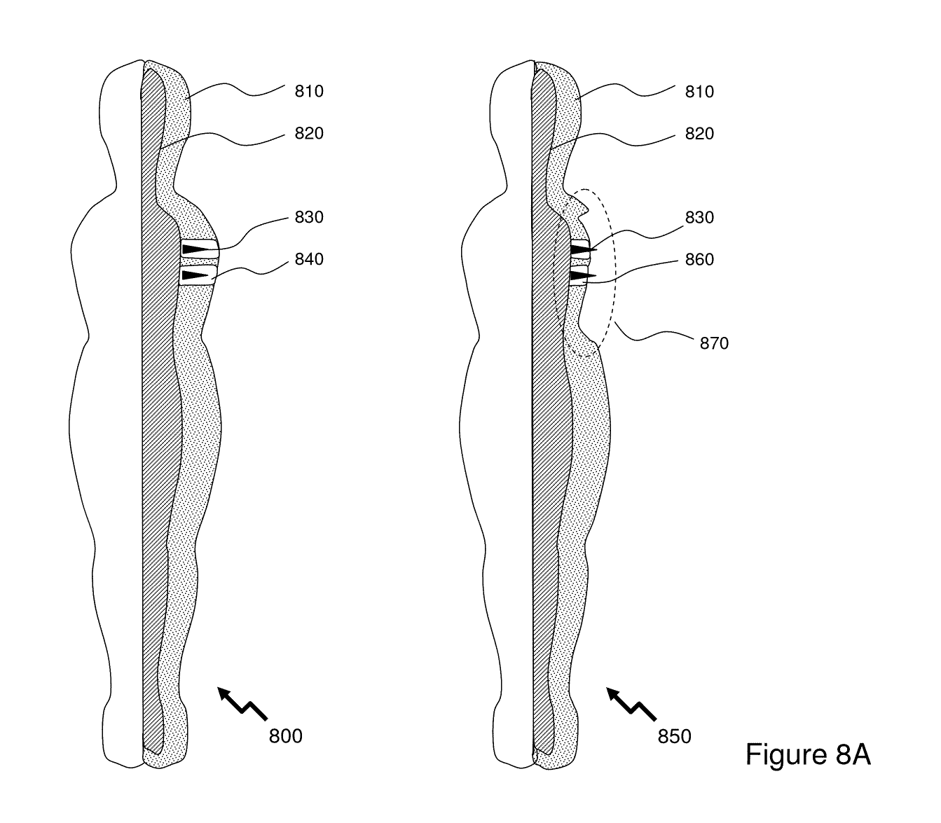

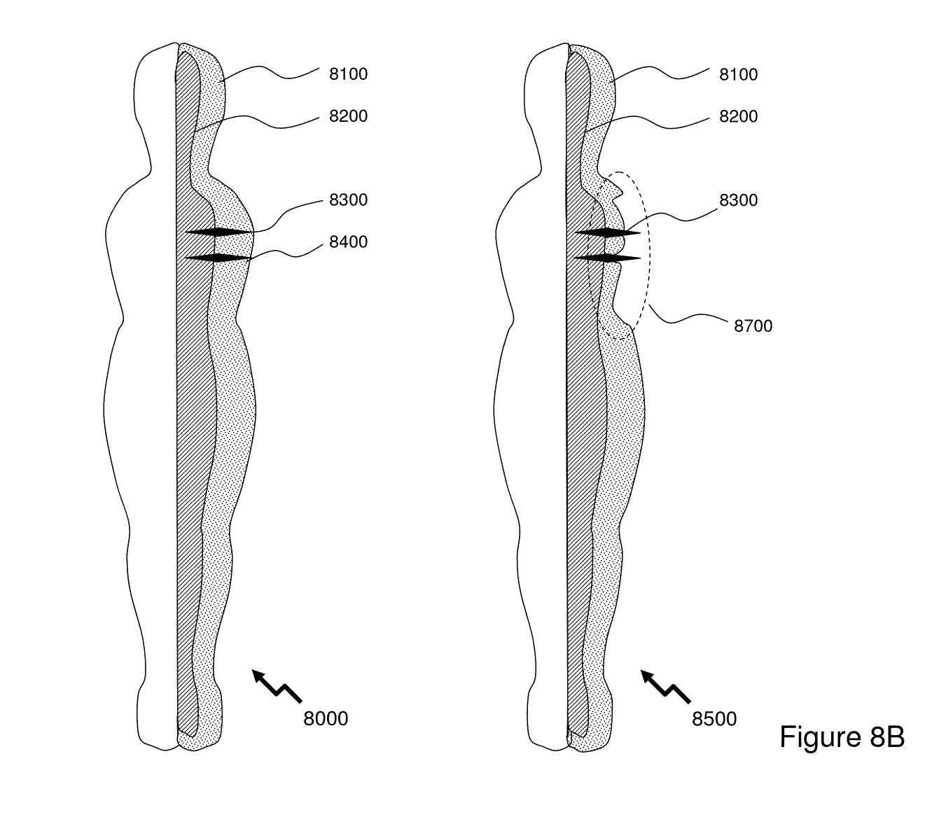

Now referring to FIG. 8A there is depicted a pain relief bobble in first and second partial cross-sections 800 and 850 incorporating needles according to an embodiment of the invention. As depicted in first partial cross-section 800 the pain relief bobble comprises an outer shell 810 and a core 820. Disposed at predetermined points are needles 830 within recesses or openings 840 in the outer shell 810. Accordingly as depicted in second partial cross-section image localized compression 850 of the outer shell 810 in the region 870 around the needles 830 results in the needles 830 being exposed above the surface of the pain relief bobble and the recesses or openings 8400 being distorted and/or collapsed. Accordingly where a patient is gripping such a pain relief bobble the needles are selectively engaged into the patient's palm, fingers, thumb etc according to their placement within the pain relief body and the patients gripping of the object. It would be evident that such an orientation is more important in this approach than the pressure based pain relief bobbles described above in respect of FIGS. 4 through 7.

The number and location of the needles may be varied according to aspects of the patient including, but not limited to, age, sex, fragility, strength, pain type, and degree of perceived pain may be considered in establishing the desired pain relief bobble for that patient.

Now referring to FIG. 8B there is depicted a pain relief bobble in first and second partial cross-sections 8000 and 8500 incorporating needles according to an embodiment of the invention. As depicted in first partial cross-section 8000 the pain relief bobble comprises an outer shell 8100 and a core 8200. Disposed at predetermined points are needles 8300 which have been inserted such that they penetrate the outer shell 8100 and partially into the core 8200. Accordingly as depicted in second partial cross-section image 8500 localized compression of the outer shell 8100 in the region 8700 around the needles 8300 results in the needles 8300 being exposed above the surface of the pain relief bobble. Accordingly where a patient is gripping such a pain relief bobble the needles are selectively engaged into the patient's palm, fingers, thumb etc according to their placement within the pain relief body and the patients gripping of the object. However, in this embodiment of the invention a single default pain relief bobble structure comprising a predetermined shape, grip features etc may be made to address a specific pain relief issue of a patient wherein a medical practitioner inserts the needles 8300. Accordingly a patient may re-use a pain relief bobble by having the pattern/quantity of needles adjusted.

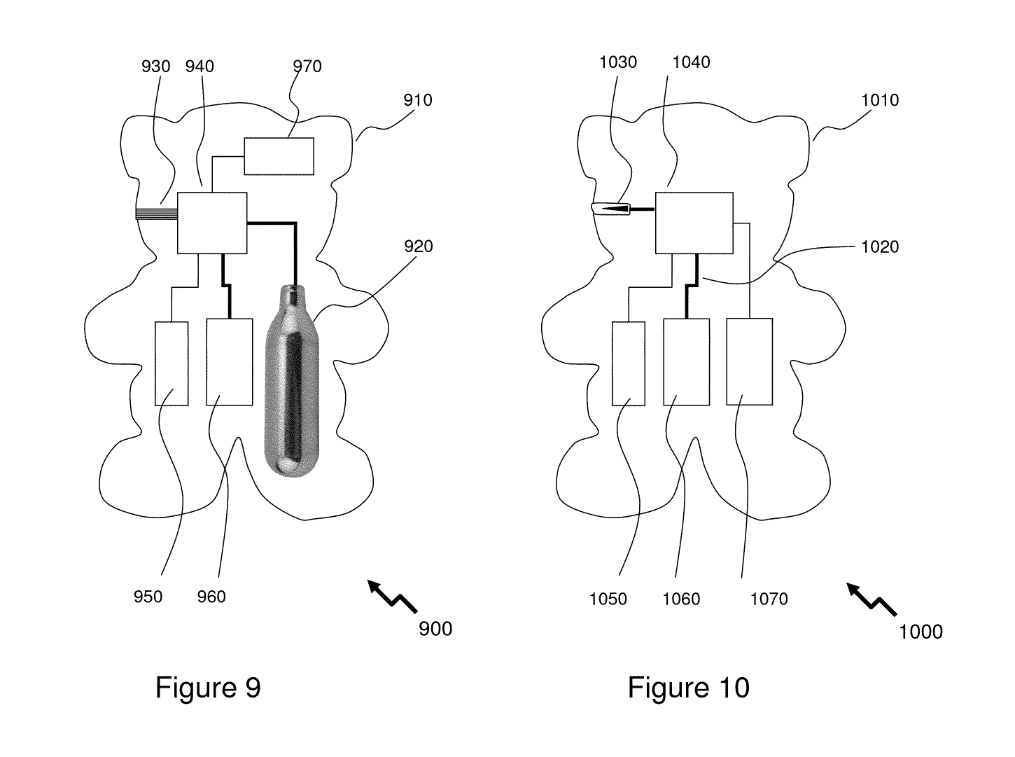

As discussed above in many instances fear, worry, etc may negatively impact a patients desire to take a medicinal treatments. Accordingly it would be beneficial to adjust their engagement to one wherein the taking of their medicine is fun or potentially even an aspect of their play activities. Accordingly there is depicted in FIG. 9 a pain relief bobble 900 incorporating a spray medication injection device 930 disguised within the pain relief bobble 900. As depicted pain relief bobble 900 comprises the spray medication injection device 930 coupled to a microfluidic control system 940 which receives medicine from a reservoir 960 and gas from a gas cartridge 920. Power for the microfluidic control system 940 being derived from a battery 970 and the trigger for activating the pain relief bobble 900 being derived from a sensor 950. For example where the pain relief bobble 900 has a deformable region then the sensor 950 may be pressure activated such that, in the instance shown, the infant or adult squeezing the “belly” of the “teddy bear” pain relief bobble 900 triggers the release of medicine from the reservoir 960 and the gas release from the gas cartridge 920. Alternatively, the sensor 950 may be a button or other form of element providing an output in response to an action. Spray injection medication device 930 may be derived from the prior art but by maintaining a small injection area the pressure may be supplied from a discrete gas cartridge 920 rather than a compressor allowing the device to be used in residential environments, medical facilities etc.

It would be evident to one skilled in the art that microfluidic control system 940 may additionally comprise electronics logging characteristics of the injection such as time and date, volume dispensed etc. Additionally, the pain relief bobble 900 may comprise a USB or other serial/parallel data port allowing this information to be retrieved from the pain relief bobble, allowing the pain relief bobble 900 to be calibrated, allowing the pain relief bobble 900 to be configured, and optionally to recharge the battery 970.

Now referring to FIG. 10 is depicted a pain relief bobble 1000 incorporating a needle injection device 1030 disguised within the pain relief bobble 1000. As depicted pain relief bobble 1000 comprises the needle injection device 1030 coupled to a microfluidic control system 1040 which receives medicine from a reservoir 1060. Power for the microfluidic control system 1040 being derived from a battery 1070 and the trigger for activating the pain relief bobble 1000 being derived from a sensor 1050. For example where the pain relief bobble 1000 has a deformable region then the sensor 1050 may be pressure activated such that, in the instance shown, the infant or adult squeezing the “belly” of the “teddy bear” pain relief bobble 1000 triggers the release of medicine from the reservoir 1060 through the needle injection device 1030.

Alternatively, the sensor 1050 may be integrated into the needle injection device 1030 such that squeezing of the pain relief bobble 1000 at that location inserts the needle into the patient's skin and triggers release of the medicine. As discussed above additional electronics and interfaces may be incorporated into the pain relief bobble 1000. Optionally, the pain relief bobble 1000 as with other embodiments of the invention with a control circuit may incorporate safety features such as preventing another activation until a predetermined period of time has elapsed, requiring that a prior activation is made such as through the pressure sensor receiving an initial significantly higher signal from an adult activating the device for subsequent use by an infant or infirm individual. Other techniques would be known within the prior art including integration of biometric sensors to establish authentication of a user priming the pain relief bobble for use.

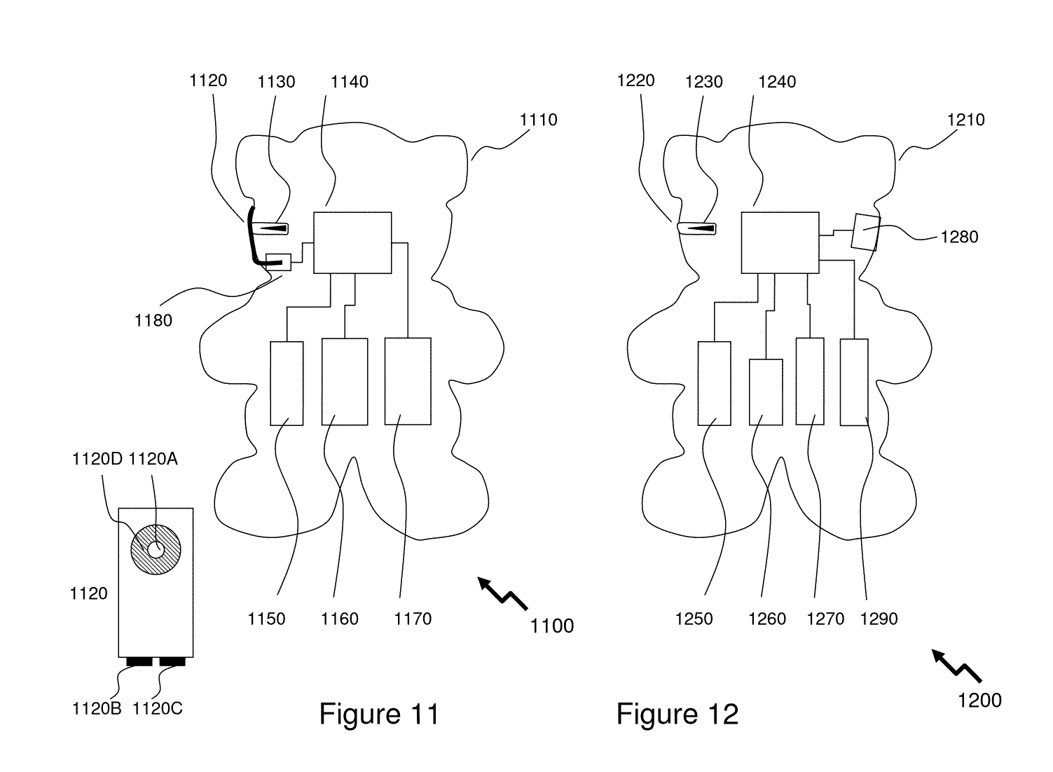

Now referring to FIG. 11 there is depicted a bobble 1100 incorporating a blood glucose testing device disguised within the bobble 1100 incorporating a needle injection device 1130 disguised within the pain relief bobble 1100 to prick the skin of a patient allowing their blood glucose level to be ascertained. As depicted bobble 1100 comprises the needle injection device 1130 which would be engaged such as described above in respect of pain relief bobbles 800 (850) and 8000 (8500) in FIGS. 8A and 8B respectively. Disposed proximate to the needle injection device 1130 is a test strip 1120 which is coupled to test strip socket 1180 and therein to controller 1140 allowing the measurement to be performed. As depicted the test strip 1120 comprises a hole 1120A allowing the needle within the needle injection device 1130 to project through into the patient and sensor region 1120D disposed radially such that the subsequent release of a small amount of blood allows the electrical measurement to be made wherein the electrical input/output to the test strip 1120 are shown by first and second contacts 1120B and 1120C respectively.

Also disposed within the bobble 1100 are display 1150, audio generator 1160, and battery 1170 coupled to the controller 1140. Accordingly the determined blood glucose value may be displayed and if the level is at a critical or dangerous level for some patients, such as the elderly or visually impaired for example, the audio generator emits an alarm signal. Optionally when the level is acceptable a different tone is generated to advise the patient that a measurement was in fact made. Likewise additional electronics and interfaces may be provided as would be evident from the disclosure above and the prior art. In the instance shown, the infant or adult squeezing the “face” of the “teddy bear” bobble 1100 triggers measurement to be performed. Disposable test strips 1120 allow the device to be reused by one patient or by multiple patients.

Now referring to FIG. 12 there is depicted a bobble 1200 incorporating a blood glucose testing device disguised within the bobble 1200 incorporating a needle injection device 1230 disguised within the pain relief bobble 1200 to prick the skin of a patient allowing their blood glucose level to be ascertained. As depicted bobble 1200 comprises the needle injection device 1230 which would be engaged such as described above in respect of pain relief bobbles 800 (850) and 8000 (8500) in FIGS. 8A and 8B respectively. Disposed proximate to the needle injection device 1230 is an optical window 1220 allowing the measurement to be performed.

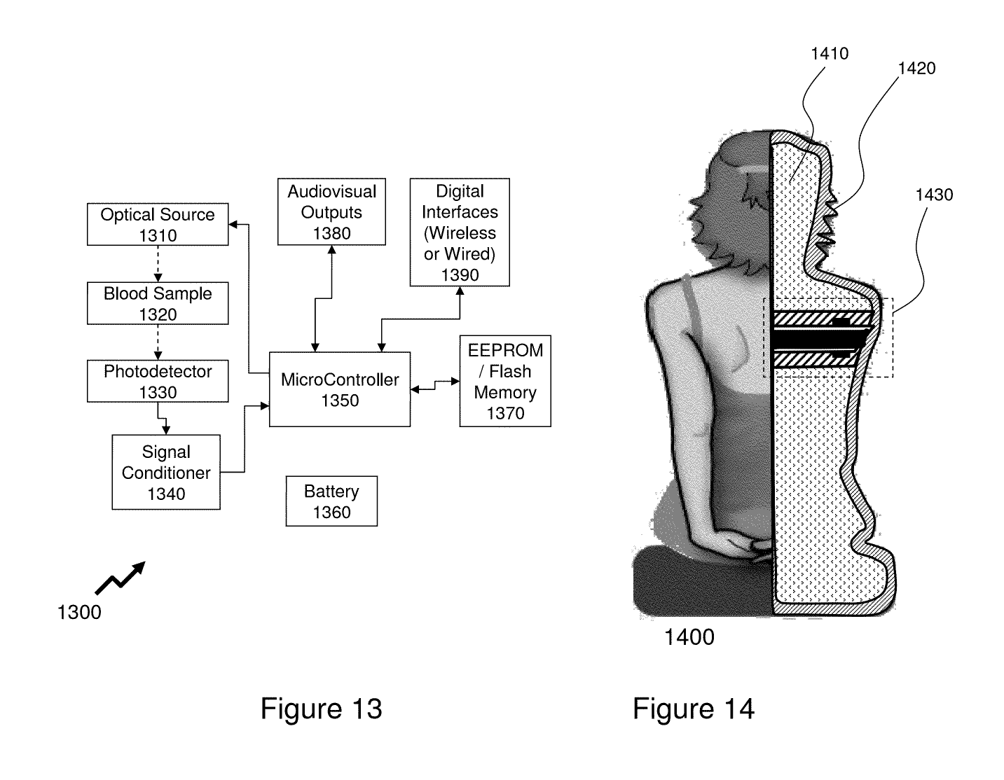

Also disposed within the bobble 1200 are display 1250, audio generator 1260, battery 1270, and Wi-Fi 1290 coupled to the controller 1240 together with optical sub-assembly, not shown for clarity, behind the optical window 1280. A simplified schematic of controller 1240 and this optical sub-assembly are described below in respect of FIG. 13. As such the determined blood glucose value may be displayed and if the level is at a critical or dangerous level for some patients, such as the elderly or visually impaired for example, the audio generator emits an alarm signal. Optionally when the level is acceptable a different tone is generated to advise the patient that a measurement was in fact made. Likewise additional electronics and interfaces may be provided as would be evident from the disclosure above and the prior art. In the instance shown, the infant or adult squeezing the “face” of the “teddy bear” bobble 1100 triggers measurement to be performed. Disposable window covers on the optical window would allow the device to be used by multiple patients and/or a single patient but are not necessary for the bobble 1200 to work as a wipe with an alcohol loaded wipe would also clean the optical window.

Now referring to FIG. 13 there is depicted a simplified schematic 1200 of the electronics for an optical blood glucose testing device according to bobble 1200 presented above in respect of FIG. 12. As shown a microcontroller 1350 is interfaced to an optical source 1310 to illuminate a blood sample 1320 such that the detected optical signal from the photodetector 1330 after conditioning by signal conditioner 1340 is coupled back to the microcontroller 1350. This resulting electrical signal is processed by the microcontroller 1350 in conjunction with calibration data stored within an EEPROM/Flash Memory 1370 to determine the blood sugar level of the patient's blood sample 1320. This data may then be coupled to audiovisual outputs 1380 as well as digital interfaces 1390 which may be wired and/or wireless. All of the electronics being coupled to a battery 1360.

Disposed within bobbles 1100 and 1200 in FIGS. 11 and 12 respectively may be a sensor or button triggering the overall measurement process which may as discussed above be integrated within the needle injection devices 1130 and 1230 respectively or be discrete. As with the pain relief bobbles described above in respect of FIGS. 4 through 8B respectively the pain relief bobbles described in FIGS. 9 through 12 may be implemented with a wide range of designs aimed at children, infants, adults, etc using different characters, animals, fantasy figures etc. Some may potentially even be themed with mature themes.

Now referring to FIG. 14 is depicted a pain relief bobble 1400 incorporating a pressure device 1430 disguised within the pain relief bobble 1400. As depicted pain relief bobble 1400 comprises the pressure device 1430 within a predetermined position and housed within the core 1420 and shell 1410. Accordingly when the user squeezes the bobble 1400 the shell 1410 and core 1420 compress faster than the central piston within the pressure device 1430. Accordingly, the pressure device 1430 applies pressure to the users palm or finger in this embodiment of the invention. However, as the piston has flanges engaging a housing forming another part of the pressure device 1430 the compression of the housing in conjunction with the piston flanges causes the piston to rotate as well as applying pressure. It would be evident to one skilled in the art multiple pressure devices 1430 may be disposed within the pain relief bobble 1400 to apply pressure to different pressure points of the user. It would be further evident that multiple pain relief bobbles may be implemented addressing different parts of the human body or that multiple pain relief bobbles for a common part of the human body may provide relief against different pains.

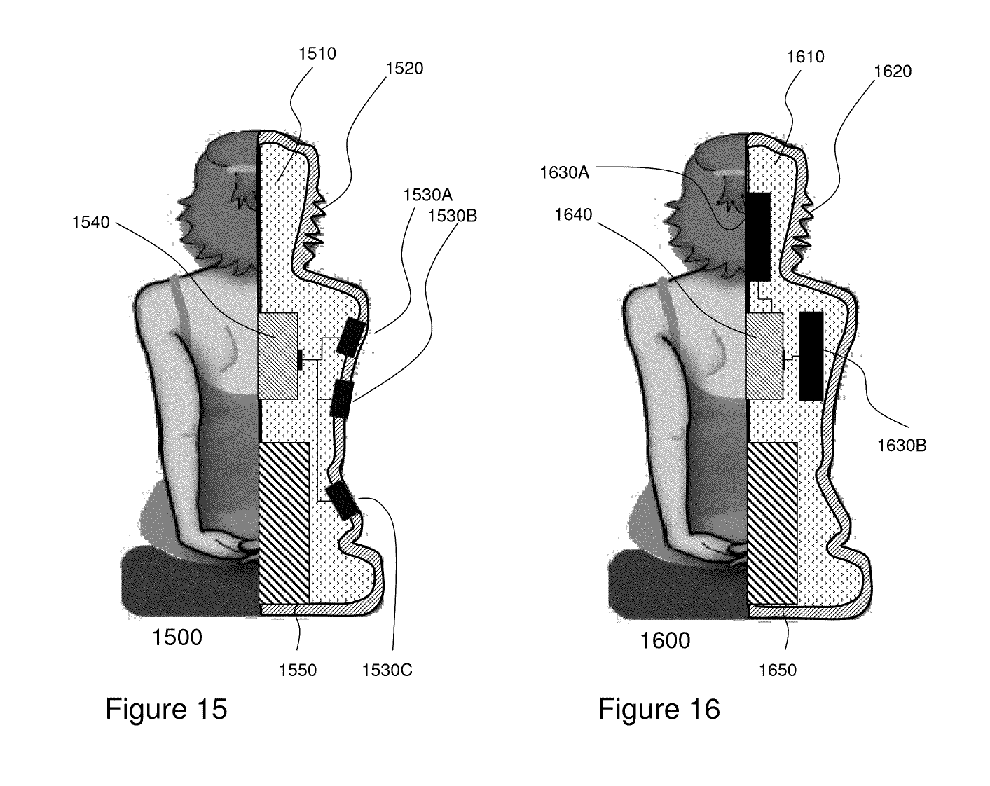

Now referring to FIG. 15 there is depicted a pain relief bobble 1500 incorporating first to third hot spots 1530A through 1530C within the outer shell 1510 of the pain relief bobble 1500. As depicted pain relief bobble 1500 comprises the first to third hot spots 1530A through 1530C within predetermined positions within the outer shell 1510. Within the core 1520 are disposed a controller 1540 and battery 1550 together with electrical connections, not labeled for clarity, providing the electrical current heating the first to third hot spots 1530A through 1530C. Optionally the controller 1540 is activated through a manual switch or a pressure switch, each of which are not shown for clarity. Where the controller 1540 is activated by a pressure switch implying that the user has squeezed the pain relief bobble 1500 then the controller 1540 may optionally implement a sequence of heating the first to third hot spots 1530A through 1530C according to a predetermined sequence.

Now referring to FIG. 16 there is depicted a pain relief bobble 1600 incorporating first and second vibration elements 1630A and 1630B within the outer shell 1610 of the pain relief bobble 1600. As depicted pain relief bobble 1600 comprises the first to third hot spots 1630A through 1630C within predetermined positions within the outer shell 1610. Within the core 1620 are disposed a controller 1640 and battery 1650 together with electrical connections, not labeled for clarity, providing the electrical current to drive the first and second vibration elements 1630A and 1630B. Optionally the controller 1640 is activated through a manual switch or a pressure switch, each of which are not shown for clarity. Where the controller 1640 is activated by a pressure switch implying that the user has squeezed the pain relief bobble 1600 then the controller 1640 may optionally implement a predetermined control sequence for the first and second vibration elements 1630A and 1630B.

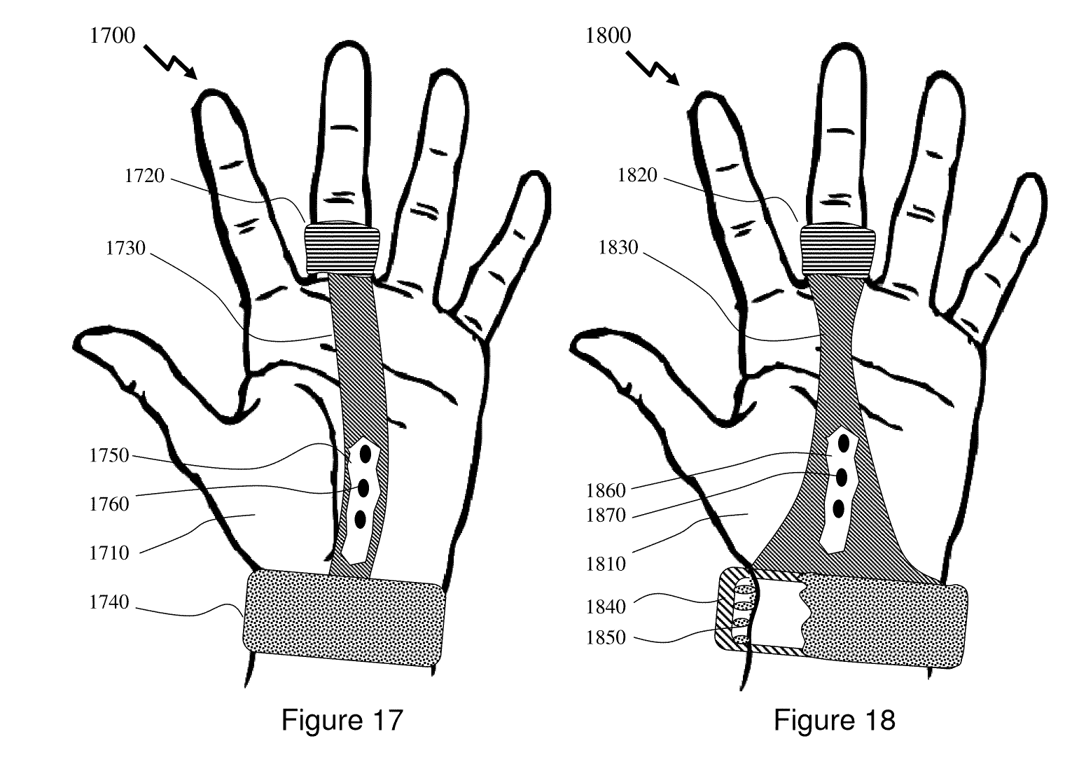

Referring to FIG. 17 there is depicted a hand massage wristband fixture 1700 for use upon a user's hand 1710 in association with a pain relief bobble according to an embodiment of the invention. The hand massage wristband fixture 1700 comprises a finger band 1720, strap 1730, and 1740. Strap 1730 as depicted in partial cross-section 1750 has disposed upon the surface against the patient's hand 1710 a plurality of elements 1760 such as described above in respect of FIGS. 4 through 7 respectively which provide pressure to those regions of the user's hand. The user in gripping the pain relief bobble, not shown for clarity, would thereby apply pressure via the pain relief bobble, to the strap 1730 and therein the plurality of elements 1760. Alternatively, the plurality of elements 1760 may be similar to those depicted supra in respect of FIGS. 8A and 8B to provide acupuncture type activation based upon the user's gripping of the pain relief bobble.

Referring to FIG. 18 there is depicted a hand massage wristband fixture 1800 for use upon a user's hand 1810 in association with a pain relief bobble according to an embodiment of the invention. The hand massage wristband fixture 1800 comprises a finger band 1820, strap 1830, and 1840. Strap 1830 as depicted in partial cross-section 1860 has disposed upon the surface against the patient's hand 1810 a plurality of elements 1870 such as described above in respect of FIGS. 4 through 7 respectively which provide pressure to those regions of the user's hand. The user in gripping the pain relief bobble, not shown for clarity, would thereby apply pressure via the pain relief bobble, to the strap 1830 and therein the plurality of elements 1870. Alternatively, the plurality of elements 1870 may be similar to those depicted supra in respect of FIGS. 8A and 8B to provide acupuncture type activation based upon the user's gripping of the pain relief bobble. In contrast to hand massage wristband fixture 1700 hand massage wristband fixture 1800 also includes a plurality of beads 1850 disposed upon the inner surface of the wristband 1840 as depicted by the partial cross-section. In this instance the material for the wristband 1840 may be selected to provide pressure and accordingly be made from an elastic material having increased contraction, and hence pressure applied, that the material employed for wristband 1740 in FIG. 17 supra.

Optionally, hand massage wristband fixtures 1700 and 1800 respectively may be implemented with a predetermined subset of the plurality of elements are connected to a controller, not shown for clarity, which may be disposed within the hand massage wristband fixture or the pain relief bobble. In the latter instance an electrical connection between the hand massage wristband fixture and pain relief bobble may be provided either as a discrete connection or one made by virtue of the gripping by the user of the pain relief bobble. These elements coupled to a controller may, for example, vibrate as well as providing pressure or acupuncture engagement to the user's hand. Alternatively, they may for example provide localized heat to the user's hand. Similarly, in hand massage wristband fixture 1800 the plurality of beads 1850 may be optionally connected to the same controller to provide for example vibration and/or heat to the patient's wrist. The plurality of beads 1850 may be activated in some embodiments of the invention, in a different or similar manner to those of the plurality of elements 1870. It would be evident that the principles of hand massage wristband fixtures 1700 and 1800 respectively may be applied with respect to other parts of the human body and pain relief bobbles designed to engage these parts, for example the foot.

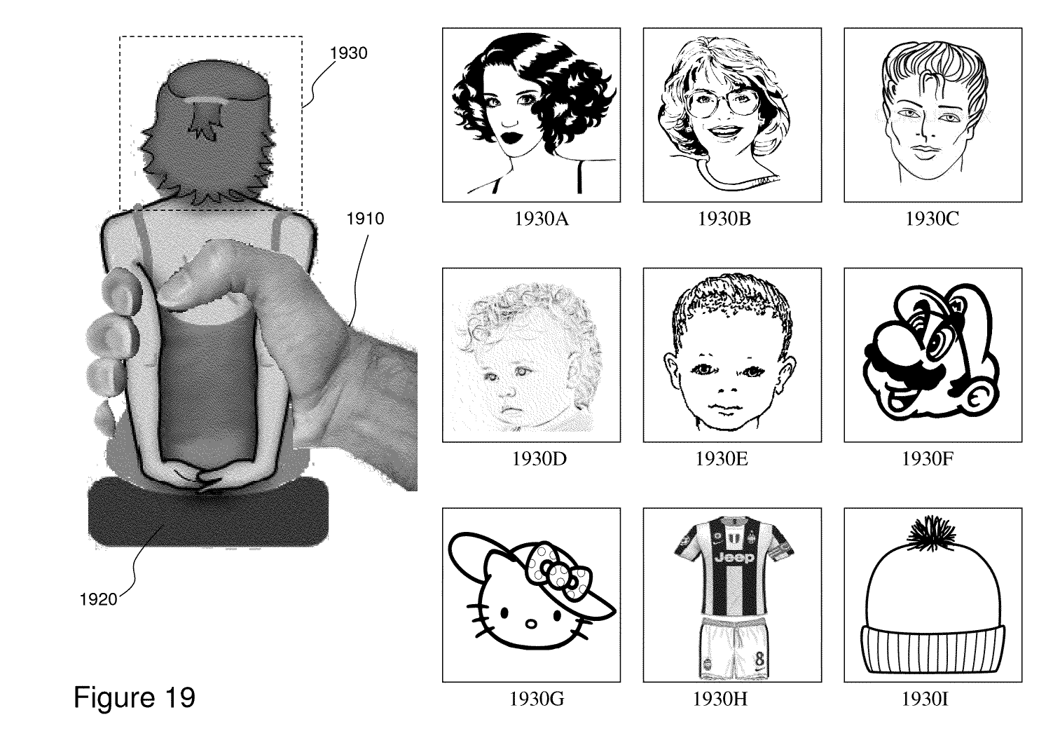

Pain relief bobbles such as described supra in respect of FIGS. 4 through 18 may be customized or personalized such as depicted in FIG. 19. According, a pain relief bobble 1920, such as described supra in respect of FIGS. 4 through 18, for gripping by the user's hand 1910 is depicted as comprising a removable head 1930. Accordingly, the user may select a head such as first and second female heads 1930A and 1930B respectively, male head 1930C, baby head 1930D, toddler head 1930E, and first and second character heads 1930F and 1930G respectively. Accordingly, a plurality of options may be provided as standard to a user or the removable head 1930 may be customized based upon a photograph of the user, a user's relative, family friend, etc. Optionally, the pain relief bobble may be molded as a single piece with the customization of the head. In either scenario additional elements may be provided to give further customization of the pain relief bobble 1920 such as headgear 1930H and clothing 1930I. For example, the user may select a favorite sports team, a hated sports team given the intention of applying pressure, or a particular style of clothing that reflects them personally or where the pain relief bobble is used as part of pain mediation in childbirth their baby.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A device comprising:
an outer shell formed from a first material characterized by a first resilience and having a first predetermined portion providing an association for an individual to a familiar object and a second predetermined portion providing a predetermined orientation of the device to a first predetermined portion of the human body when placed against a second predetermined portion of the human body; and
at least one member of a plurality of members, the at least one member formed from a second material characterized by a second resilience greater than the first resilience and being positioned in a first predetermined position within the device so as to engage a predetermined portion of the first predetermined portion of the human body.

2. The device according to claim 1 wherein,
the at least one member is disposed at least one of inside the outer shell and embedded into the outer shell.

3. The device according to claim 1 wherein,
the at least one member is disposed within a filler filling a predetermined portion of the outer shell.

4. The device according to claim 1 wherein,
the outer shell comprises a plurality of recesses disposed so as to allow the at least one member to be positioned in a plurality of predetermined positions in order to engage predetermined portions of the first predetermined portion of the human body, the plurality of first predetermined positions including the first predetermined position.

5. The device according to claim 1 wherein,
compression of a predetermined portion of the outer shell against the second predetermined region of the human body results in the at least one member of the plurality of members applying pressure to the predetermined portion of the first predetermined portion of the human body.

6. A device comprising:
an outer shell formed from a first material characterized by a first resilience and having a first predetermined portion providing an association for an individual to a familiar object and a second predetermined portion providing a predetermined orientation of the device to a first predetermined portion of the human body when placed against a second predetermined portion of the human body;
at least one member of a plurality of members, the at least one member formed from a second material characterized by a second resilience greater than the first resilience and being positioned in a first predetermined position within the device so as to engage a predetermined portion of the first predetermined portion of the human body; and
at least one needle disposed in a predetermined position with respect to the at least one member of the plurality of members.

7. The device according to claim 6 wherein,
the at least one member is at least one of disposed inside the outer shell and forms a filler filling a predetermined portion of the outer shell.

8. The device according to claim 6 wherein,
the outer shell comprises a plurality of recesses disposed so as to allow the at least one member to be positioned in a plurality of predetermined positions in order to engage predetermined portions of the first predetermined portion of the human body, the plurality of first predetermined positions including the first predetermined position.

9. The device according to claim 6 wherein,
compression of a predetermined portion of the outer shell against the second predetermined region of the human body results in the at least needle of the plurality of needles at least one of applying pressure to and penetrating into the predetermined portion of the first predetermined portion of the human body.

10. A device comprising:
an outer shell formed from a first material characterized by a first resilience and having a first predetermined portion providing an association for an individual to a familiar object and a second predetermined portion providing a predetermined orientation of the device to a first predetermined portion of the human body when placed against a second predetermined portion of the human body;
at least one member of a plurality of members, the at least one member formed from a second material characterized by a second resilience greater than the first resilience and being positioned in a first predetermined position within the device so as to engage a predetermined portion of the first predetermined portion of the human body; and
at least one needle of a plurality of needles disposed in a predetermined position with respect to the at least one member of the plurality of members;
a pressure activated sensor;
a microcontroller connected to the pressure activated sensor; and
a microfluidic delivery system disposed within the outer body connected to the needle and to a reservoir containing a fluid, the microfluidic delivery system controlled by the microcontroller.

11. The device according to claim 10 wherein,
the at least one member is at least one of disposed inside the outer shell and forms a filler filling a predetermined portion of the outer shell.

12. The device according to claim 10 wherein,
the outer shell comprises a plurality of recesses disposed so as to allow the at least one member to be positioned in a plurality of predetermined positions in order to engage predetermined portions of the first predetermined portion of the human body, the plurality of first predetermined positions including the first predetermined position.

13. The device according to claim 10 wherein,
compression of a predetermined portion of the outer shell against the second predetermined region of the human body results in the at least needle of the plurality of needles at least one of applying pressure to and penetrating into the predetermined portion of the first predetermined portion of the human body.

14. The device according to claim 10 wherein,
compression of a predetermined portion of the outer shell against the second predetermined region of the human body results in the pressure activated sensor generating a signal to the microcontroller thereby triggering transfer of a predetermined amount of the fluid through the at least needle of the plurality of needles.

15. The device according to claim 10 further comprising;
a gas cartridge storing a gas under pressure coupled to the microfluidic system.

16. The device according to claim 15 wherein,
compression of a predetermined portion of the outer shell against the second predetermined region of the human body results in the pressure activated sensor generating a signal to the microcontroller thereby triggering transfer of a first predetermined amount of the fluid from the reservoir and a second predetermined amount of the gas from the gas cartridge thereby dispensing the fluid under pressure.

17. The device according to claim 10 further comprising;
an atomizer forming part of the microfluidic circuit for atomizing fluid withdrawn from the reservoir.
